(19) United States
(12) Patent Application Publication
(10) Pub. No.: US 2022/0401324 A1
(43) Pub. Date: Dec. 22, 2022

(54) COSMETIC PREPARATION WITH ANISIC ACID AND LEVULINIC ACID, HAVING SELECTIVE ANTIMICROBIAL EFFECT

(71) Applicant: BEIERSDORF AG, Hamburg (DE)

(72) Inventors: Bernd TRAUPE, Hamburg (DE); Helga BIERGIESSER, Reinbek (DE); Andreas FIRYN, Schwarzenbek (DE); Dominik GOEDDERTZ, Hamburg (DE); Lucia Zanforlin TREDE, Hamburg (DE); Luis Arturo Carbajal AVILA, Hamburg (DE); Birthe KOERBL, Hamburg (DE); Heike FOELSTER, Hamburg (DE); Tina HAMANN, Brodersby-Goloft (DE); Petra SCHOENDIENST, Tornesch (DE); Daniel RICHTER, Norderstedt (DE)

(21) Appl. No.: 17/756,089

(22) PCT Filed: Nov. 20, 2020
(86) PCT No.: PCT/EP2020/082863

§ 371 Date: May 17, 2022

(30) Foreign Application Priority Data
Nov. 20, 2019 (DE) 10 2019 217 898.7
Sep. 4, 2020 (EP) 20194680.3

(51) Int. Cl. A61K 8/368; A61K 8/365; A61K 8/04; A61K 8/02; A61P 31/04; A61P 17/10; A61Q 17/00; A61Q 19/00

(52) U.S. Cl. CPC A61K 8/368; A61K 8/365; A61K 8/042; A61K 8/0229; A61P 31/04; A61P 17/10; A61Q 17/005; A61Q 19/005

(57) ABSTRACT

The invention relates to cosmetic and dermatological preparations which comprise a combination of levulinic acid and anisic acid. The preparations according to the invention are particularly suitable for use on skin. The preparations have a selective antibacterial effect on skin by specifically controlling anaerobic bacteria of the skin flora while having little or no bactericidal effect on aerobic bacteria. The compositions according to the invention are therefore suitable for cosmetic skin care and for the dermatological treatment of certain skin disorders, such as acne.

The invention relates to cosmetic and dermatological preparations comprising a combination of levulinic acid and anisic acid. The preparations according to the invention are especially suitable for use on the skin. The preparations exhibit a selective antibacterial effect on the skin by specifically controlling anaerobic bacteria of the skin flora, while having no bactericidal effect, or only a slight bactericidal effect, against aerobic bacteria. Accordingly, the compositions according to the invention are suitable for cosmetic skin care and also for dermatological treatment of certain skin disorders, such as, for example, acne.

### BACKGROUND OF THE INVENTION

The microbiome of human skin is a complex mixture of different groups of microorganisms: anaerobic bacteria, such as Cutibacterium acnes (C. acnes), colonize the skin in the immediate vicinity of aerobic bacteria, such as Staphylococcus epidermidis (S. epidermidis) and Staphylococcus hominis (S. hominis), and fungi.

It is known that the interactions between S. epidermidis and C. acnes is of particular importance for skin health. S. epidermidis is an aerobically growing, spherical, Gram-positive bacterium which is catalase-positive and coagulase-negative. C. acnes is an anaerobically growing, Gram-positive bacterium. It is suspected that both microorganisms occur on healthy skin in a relatively stable balance and that a disturbance of this balance, so-called dysbiosis, can have an adverse effect on the condition of the skin by causing skin blemishes or even skin disorders or at least promoting the development thereof. For the treatment or prevention of such skin blemishes or skin disorders, it is therefore of particular importance to provide means which make it possible to specifically alter the ratio of anaerobic to anaerobic microorganisms of the skin flora, and in particular the ratio of S. epidermidis and C. acnes. Since especially the increased growth of anaerobic bacteria, such as, for example, P. acnes, may be associated with the development of skin disorders, what are especially needed are preparations which are active against anaerobic bacteria in a selective manner.

Anisic acid is a methoxylated benzoic acid (4-methoxybenzoic acid) which is bacteriostatic and which is also considered to have an anti-inflammatory effect and also a fungicidal effect. Anisic acid is used in, inter alia, cosmetic products as a preservative in order to avoid contamination of the products by bacteria or fungi. For this purpose, anisic acid is added to said products in an amount of 0.05-0.3%. Levulinic acid, which is also called 4-oxopentanoic acid or 4-oxovaleric acid, is a chemical compound which belongs to the γ-keto acids. Declared internationally as “levulinic acid”, the acid and the salts thereof are in demand in the field of skin care as a fragrance additive, since the typical odor thereof is pleasantly reminiscent of caramel or vanilla. Furthermore, it is known that levulinic acid has an antibacterial and fungicidal effect. This means that this substance is also of interest as a skin care product for blemished skin. Since levulinic acid is not declared as a preservative, it can be ideally used for preserving natural cosmetics. There has been ample description of use of levulinic acid as a preservative in cosmetic products, the substance being added to the products to be preserved in an amount of 0.3-1.0%.

Preservatives in cosmetics are regularly used in order to protect the product from contamination by bacteria and fungi. The microbiological stability of cosmetics is a crucial factor for shelf life and for protecting the health of consumers. The European Cosmetics Regulation defines preservatives as substances which are used exclusively or mainly for inhibiting the development of microorganisms in cosmetic products. The substances allowed for this purpose are listed in the corresponding positive list in Annex V of the EU Cosmetics Regulation.

The use of anisic acid and levulinic acid as preservatives has been described in the prior art. EP 1 541 124 A2 describes a synergistic effect between two active ingredients used for preservation, namely anisic acid and C6-14-fatty acid monoglycerides. The mixture is intended to exhibit antimicrobial activity which can be used for preservation of cosmetic formulations. The formulations therefore give rise to a broad antimicrobial composition with, at the same time, good skin compatibility. EP 2 735 301 A1 discloses the combination of levulinic acid, the sodium salt of levulinic acid (sodium levulinate) and anisic acid for preservation of a cosmetic product.

Whereas anisic acid and levulinic acid have extensively proven themselves in the preservation of cosmetic products, the use of these substances to specifically alter the composition of the skin flora is so far unknown. As explained above, there is a need for cosmetic and dermatological compositions which contribute to improving the general condition of the skin and the health of the skin by making a specific intervention in the composition of the skin flora. At the same time, the compositions should have good skin compatibility.

### DESCRIPTION OF THE INVENTION

The present invention is based on the surprising finding that anisic acid can be used in combination with levulinic acid to selectively reduce the concentration of anaerobic bacteria in the skin flora, especially C. acnes. Consequently, the invention relates to the use of a combination of anisic acid and levulinic acid for reduction of anaerobic bacteria and simultaneous preservation of aerobic bacteria on the skin. The combination according to the invention thus allows a selective antibacterial effect to develop on the skin after application to the skin.

In a first aspect, the invention provides a composition for specific alteration of the skin flora, wherein the composition comprises the following:
(i) anisic acid or an antimicrobially active salt thereof, and
(ii) levulinic acid or an antimicrobially active salt thereof.

Consumption measured compositions preferably contain anisic acid. However, salts of anisic acid can also be used, provided that they have sufficient antimicrobial activity. Salts of anisic acid that are suitable for use in the context of the present invention preferably have antimicrobial activity corresponding to the activity of anisic acid to an extent of at least 50% in an appropriate in vitro assay. This means that, when using equal amounts of anisic acid and the salt of anisic acid to determine antimicrobial activity in an in vitro assay, the salt of anisic acid must have at least half the antimicrobial activity compared to anisic acid. Suitable salts for use in the context of the present invention are sodium anisate and potassium anisate.

Salts of levulinic acid that are suitable for use in the context of the present invention preferably have an antimicrobial activity corresponding to the activity of levulinic acid to an extent of at least 50% in an appropriate in vitro assay. This means that, when using equal amounts of levulinic acid and the salt of levulinic acid to determine antimicrobial activity in an in vitro assay, the salt of levulinic acid must have at least half the antimicrobial activity compared to levulinic acid. Suitable salts for use in the context of the present invention are sodium levulinate and potassium levulinate.

More particularly, the present invention provides a composition for specific alteration of the skin flora, wherein the composition
(i) more than 0.3% (w/w) anisic acid or an antimicrobially active salt thereof, and
(ii) more than 1.0% (w/w) levulinic acid or an antimicrobially active salt thereof.

In a particularly preferred embodiment, the composition comprises at least 0.5% (w/w) anisic acid or an antimicrobially active salt thereof, and at least 1.5% (w/w) levulinic acid or an antimicrobially active salt thereof. Thus, the amount of anisic acid or anisic acid salt in the composition is preferably at least 0.75%, at least 1.0%, at least 1.5%, at least 2.0%, at least 2.5%, at least 3.0%, at least 3.5%, at least 4.5% or at least 5.0% (w/w). The amount of levulinic acid or levulinic acid salt in the composition is preferably at least 2.0%, at least 2.5%, at least 3.0%, at least 3.5%, at least 4.5% or at least 5.0% (w/w).

In other words, the composition of the present invention preferably comprises between 0.5% and 5.0% (w/w) anisic acid or an antimicrobially active salt thereof, and between 1.5% and 5.0% (w/w) levulinic acid or an antimicrobially active salt thereof. Particular preference is given to a composition comprising between 1.0% and 4.0% (w/w) anisic acid or anisic acid salt and between 2.0% and 4.0% (w/w) levulinic acid or levulinic acid salt. Even greater preference is given to a composition comprising between 1.5% and 3.0% (w/w) anisic acid or anisic acid salt and between 2.0% and 3.0% (w/w) levulinic acid or levulinic acid salt. Even greater preference is given to a composition comprising between 2.0% and 3.0% (w/w) anisic acid or anisic acid salt and between 2.5% and 3.0% (w/w) levulinic acid or levulinic acid salt.

The compositions according to the invention can be present as a unitary formulation or else as components which are combined together immediately before application to the skin. In the latter case, the above quantitative data are to be applied to the composition that results from combining the components immediately before application. The present invention also encompasses compositions in which the two antimicrobially active components, namely anisic acid or the salt thereof and levulinic acid or the salt thereof, are present as separate formulations which are applied to the skin one after the other. For instance, the two antimicrobially active components can be applied to the skin in a staggered manner at an interval of about 5 minutes, 10 minutes, 15 minutes, 20 minutes, 24 minutes or 30 minutes.

The compositions of the present invention can be present in various forms. Examples of preferred preparation forms are a solution, a suspension, a water-in-oil (W/O) emulsion or oil-in-water (O/W) emulsion, or a multiple emulsion, for example water-in-oil-in-water (W/O/W) emulsion or oil-in-water-in-oil (O/W/O) emulsion, a hydrodispersion or lipodispersion, or else an aerosol. Preferably, the composition according to the invention is present as emulsions containing not only the antimicrobially active components, but also other substances such as, for example, fats, oils, waxes and/or other lipids and also water and one or more emulsifiers, as are customarily used for a formulation of this kind. Such emulsions can advantageously be formulated as a cream or lotion. Furthermore, it is also possible and advantageous to introduce the combinations of anisic acid and levulinic acid according to the invention into aqueous systems or surfactant preparations for cleaning of the skin and/or hair.

It is of course known to a person skilled in the art that cosmetic and therapeutic compositions are usually not conceivable without the customary excipients and additives. These include, for example, consistency enhancers, fillers, fragrances, dyes, emulsifiers, additional active ingredients such as vitamins or proteins, light stabilizers, stabilizers, insect repellents, alcohol, water, salts, and also antimicrobially, proteolytically or keratolytically active substances, etc., provided that the use thereof does not significantly inhibit or is not contrary in some other way to the antimicrobial activity of the combination according to the invention. The combinations of anisic acid and levulinic acid according to the invention can advantageously be incorporated into customary cosmetic and dermatological preparations, which in turn can be present in various forms. The compositions can, for example, be present as a cream, lotion, gel, ointment, paste or solid stick.

The above-described compositions are particularly suitable for the non-therapeutic, cosmetic treatment and/or the therapeutic treatment of the skin, especially the facial skin of a human. In a particular embodiment, the invention relates to a composition as described above for use in a method for treating a skin disorder. The skin disorder is preferably dermatitis. Particular preference is given to treating acne (acne vulgaris) with the compositions herein above. The therapeutic effect of the compositions according to the invention advantageously arises from the specific alteration of the concentration of anaerobic bacteria on the skin. The concentration of said anaerobic bacteria is reduced, though the concentration of anaerobic bacteria in the same habitat is not reduced or not significantly reduced. This results in a shift in the ratio of anaerobic to aerobic bacteria on the skin, and in particular a shift in the ratio of C. acnes to S. epidermidis.

The compositions according to the invention are additionally also suitable for non-therapeutic, cosmetic use. Such non-therapeutic, cosmetic use can serve for the selective reduction of the number of anaerobic bacteria on the skin, and in particular the selective reduction of the number of C. acnes. This can eliminate or avoid skin blemishes. In particular, blockages of sebaceous glands, as occur in the case of comedones for example, can be avoided. The non-therapeutic, cosmetic use can thus considerably improve the visual appearance of the skin. In another embodiment, the invention additionally also relates to the non-therapeutic use of a composition of the preparation according to the invention as defined above for skin care in the event of acne.

The compositions can be applied to the skin once or multiple times as part of the therapeutic or non-therapeutic, cosmetic treatment. In one embodiment, the compositions according to the invention are formulated for daily application to the skin. In an alternative embodiment, the compositions according to the invention are formulated for single application to the skin. Surprisingly, the combination of anisic acid and levulinic acid in the compositions according to the invention acts selectively, i.e., the anaerobic bacteria, such as C. acnes, are reduced on the skin and the aerobic bacteria, such as S. epidermidis, are not attacked or not significantly attacked.

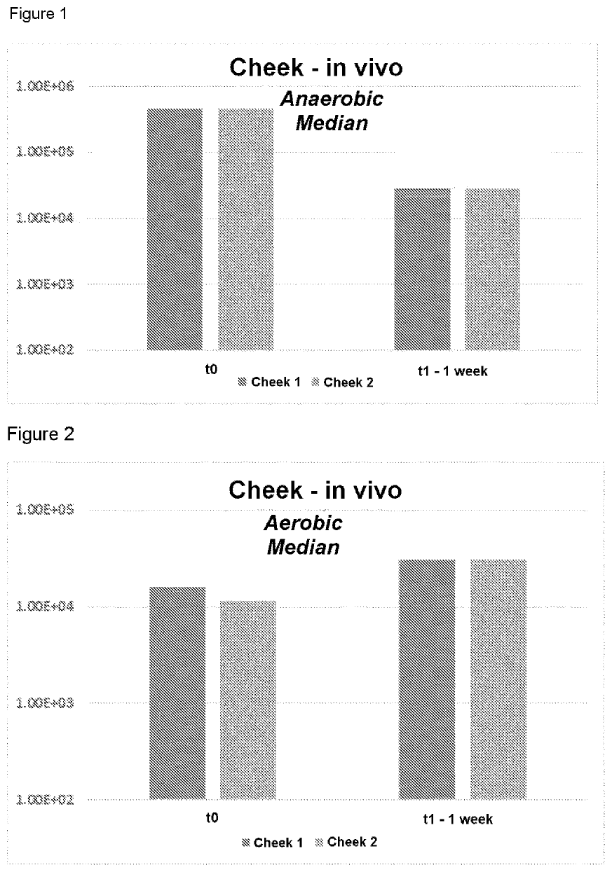

The advantage of the compositions according to the invention lies in the high antimicrobial selectivity. The compositions specifically lead to a significant drop in the cell count of anaerobic bacteria colonizing the skin, especially C. acnes. At the same time, the compositions do not have an inhibitory effect, or have only a slight inhibitory effect, on A aerobic bacteria, especially S. epidermidis, the number of which on the skin is not reduced to a significant extent. As illustrated in the examples described below and shown in FIGS. 1-2, the use of the compositions according to the invention leads to a 90% drop in the anaerobic bacteria, whereas the aerobic bacteria even further multiplied over the test period.

In the present case, it is preferred that the number of anaerobic bacteria in a specific skin area after application of the composition according to the invention declines by at least 50%, at least 60%, at least 70%, at least 80%, at least 90% or at least 95% compared to the cell count in the skin area in question before application of the composition. Furthermore, it is preferred that the number of aerobic bacteria in said area of skin declines, at the same time, by not more than 25%, not more than 20%, not more than 15%, not more than 10%, not more than 5% or less compared to the cell count in the skin area in question before application of the composition.

In the present case, it is particularly preferred that the number of C. acnes in a specific skin area after application of the composition according to the invention declines by at least 50%, at least 60%, at least 70%, at least 80%, at least 90% or at least 95% compared to the cell count in the skin area in question before application of the composition. Furthermore, it is preferred that the number of S. epidermidis in said skin area declines, at the same time, by not more than 25%, not more than 20%, not more than 15%, not more than 10%, not more than 5% or less compared to the cell count in the skin area in question before application of the composition.

The efficacy of the compositions according to the invention has been shown in various tests, which are described in the examples which follow. To this end, in one test, bacteria were obtained from the cheek by means of a swabbing technique and incubated aerobically and anaerobically (i.e., with reduced oxygen content). After 14 days of use, bacteria were again taken from the test subjects. A surprising picture become apparent after all test subject data were evaluated. The aerobically cultured bacteria showed no abnormalities, whereas the anaerobically cultured bacteria exhibited a significant reduction in the bacterial cell count of more than 90% after just one week. Further in vitro tests were able to confirm this selective behavior.

### EXAMPLES

The examples below are intended to illustrate the present invention without limiting it. Unless otherwise stated, all quantitative data, proportions and percentages are based on the weight and the total amount or the total weight of the respective preparations and compositions.

### Example 1: Preparation of Bacterial Suspensions and Growth Experiments

S. epidermidis (DSM 1798) was taken from a cryotube (EN 12353), streaked on a CASO agar plate and incubated at 37° C. for 24 h. This was followed by creating a second passage. 10 ml of BHI medium and 5 g of glass beads (4 mm in diameter) were inoculated with the second passage and shaken for 3 min. Thereafter, this batch was adjusted to an OD600 of 0.1 and then diluted 1:10 with diluent. This bacterial suspension was then used for the growth assay.

Test—Growth Experiment with S. epidermidis

10 μL of bacterial suspension were added to each well of a 24-well plate and left to dry completely (at least 45 min in a clean bench). After drying, 10 mg of the formulation to be tested (see Table 1) were pipetted onto the dried suspension and spread in the well using a T-shaped spreader. The control was left untreated. The plate was stored without the lid in a humidity incubator at 37° C. and 90% air humidity for the incubation period (6 h/24 h). At the end of the incubation time, 500 μL of rinse-off buffer were added to each well and rinse-off was carried out in an ultrasonic bath for 1 min. 100 μL of the supernatant obtained were added to 900 μL of neutralization agent and then plated out on CASO agar using a spiral plater. The agar plates were incubated at 37° C. for 24 h and the colonies were then counted using an I+L Countermat.

C. acnes (DSM 1897) was streaked from a cryotube (EN 12353) on an agar plate (COST agar) and incubated in an anaerobic box at 37° C. for 5 days. 10 ml of anaerobic medium in a CELLSTAR CELLreactor tube were inoculated with this culture and incubated in the anaerobic box for 18 h. Thereafter, this batch was adjusted to an OD600 of 0.5 and then diluted 1:10 with anaerobic medium. This bacterial suspension was then used for the growth assay.

Test—Growth Experiment with C. acnes

10 μL of bacterial suspension were added to each well of a 24-well plate and left to dry completely (at least 45 min in a clean bench). After drying, 10 mg of the formulation to be tested (see Table 1) were pipetted onto the dried suspension and spread in the well using a T-shaped spreader. The control was left untreated. The plate was stored without the lid in an anaerobic box (lined with cellulose+50 mL of water) in an incubator at 37° C. for the incubation period (6 h/24 h). At the end of the incubation time, 500 μL of rinse-off buffer were added to each well and rinse-off was carried out in an ultrasonic bath for 1 min. 100 μL of the supernatant obtained were added to 900 μL of neutralization agent and then plated out on COST agar using a spiral plater. The agar plates were incubated at 37° C. for 5 days and the colonies were then counted using an I+L Countermat.

The reduction factor was determined as part of the evaluation. The reduction factor is the factor by which the cell count (CFU) of the different time points is reduced compared to the TO control or to one another.

$$\frac{CFU ( Control )}{CFU ( Test substance )} = Reduction factor$$

The study design is designed so that antibacterial efficacy of the test formulations is demonstrated by comparison with the untreated control.

TABLE 1
Formulations tested and results of the growth experiments

| | Formulation No. | | | | | |
|---|---|---|---|---|---|---|
| | 1 | 2 | 3 | 4 | 5 | 6 |
| INCI | % | % | % | % | % | % |
| p-Anisic Acid | 0.1 | 0.1 | 0.1 | 0.1 | 0.1 | 0.1 |
| Levulinic Acid (Aqua/Glycerin/ | 0.5 | 0.5 | 0.5 | 0.5 | 0.5 | 0.5 |
| Sodium Levulinate) | | | | | | |
| Glycerin + Arctium Lappa Fruit Extract | 1.2 | 1.2 | 1.2 | 0 | 0 | 0 |
| Ethylhexylglycerin | 0.1 | 0.1 | 0 | 0.1 | 0.2 | 0.1 |
| Decylene Glycol | 0 | 0.1 | 0.1 | 0.1 | 0 | 0 |
| Sodium Hyaluronate + Aqua | 0 | 0 | 0.1 | 0 | 0 | 0 |
| Maltodextrin + Chamomilla Recutita | 0 | 0 | 0 | 0.6 | 0 | 0 |
| Flower Extract | | | | | | |
| Dicaprylyl Ether | 2.8 | 3.5 | 0 | 3.5 | 2.8 | 2.8 |
| Simmondsia Chinensis Seed Oil | 3.5 | 3.5 | 0 | 3.5 | 3.5 | 3.5 |
| Butyrospermum Parkii Butter | 2 | 2 | 0 | 2 | 3.5 | 2 |
| Prunus Amygdalus Dulcis Oil | 3 | 3.5 | 0 | 3.5 | 5 | 3 |
| Coco-Caprylate/Caprate | 1.2 | 0 | 0 | 0 | 0 | 1.2 |
| Argania Spinosa Kernel Oil | 0.1 | 0.1 | 0.1 | 0 | 0.1 | 0 |
| Caprylic/Capric Triglyceride | 0 | 1 | 0 | 1 | 0 | 0 |
| Brassica Campestris Seed Oil | 0 | 0 | 6 | 0 | 0 | 0 |
| Glyceryl Stearate | 2 | 2 | 0 | 2 | 2 | 2 |
| Glyceryl Caprylate | 0.3 | 0.3 | 0.3 | 0.3 | 0.3 | 0.3 |
| Sodium Stearoyl Glutamate | 0.3 | 0.3 | 0 | 0.3 | 0.3 | 0.3 |
| Sodium Cetearyl Sulfate | 0 | 0 | 0.2 | 0 | 0 | 0 |
| Parfum | 0.35 | 0.35 | 0.35 | 0 | 0.3 | 0.3 |
| Glycerin | 8.4 | 8.4 | 9 | 8.4 | 8.4 | 8.4 |
| Cetearyl Alcohol | 5 | 5 | 0 | 5 | 5 | 5 |
| Xanthan Gum | 0.3 | 0.3 | 0.2 | 0.3 | 0.3 | 0.3 |
| Cetyl Alcohol | 0 | 0 | 3 | 0 | 0 | 0 |
| Hydroxypropyl Starch Phosphate + | 0 | 0 | 3 | 0 | 0 | 0 |
| Aqua | | | | | | |
| Alcohol Denat. + Aqua | 4 | 4 | 4 | 4 | 4 | 4 |
| Aqua | to 100 | to 100 | to 100 | to 100 | to 100 | to 100 |
| Reduction factors | 1 | 2 | 3 | 4 | 5 | 6 |
| Staphylococcus epidermidis after 6 h | 1 | 1 | 10 | 1 | 1 | 1 |
| Cutibacterium acnes after 6 h | 40 | 134 | 1651 | 214 | 813 | 71 |

The counting showed that the number of aerobic S. epidermidis bacteria was not affected or not significantly affected by the formulation containing anisic acid and levulinic acid. In the S. epidermidis batches, only small reduction factors were measured. By contrast, the number of anaerobic C. acnes bacteria was significantly reduced by the formulations.

### Example 2: ATA Test

The ATA test is used to determine the antibacterial efficacy of cosmetic formulations after twice daily application. The test period is generally 1 week. Application of the test formulation and measurement of efficacy are done in the area relevant to the consumer, the cheek. The target variable is the microbial count for aerobic and anaerobic bacteria.

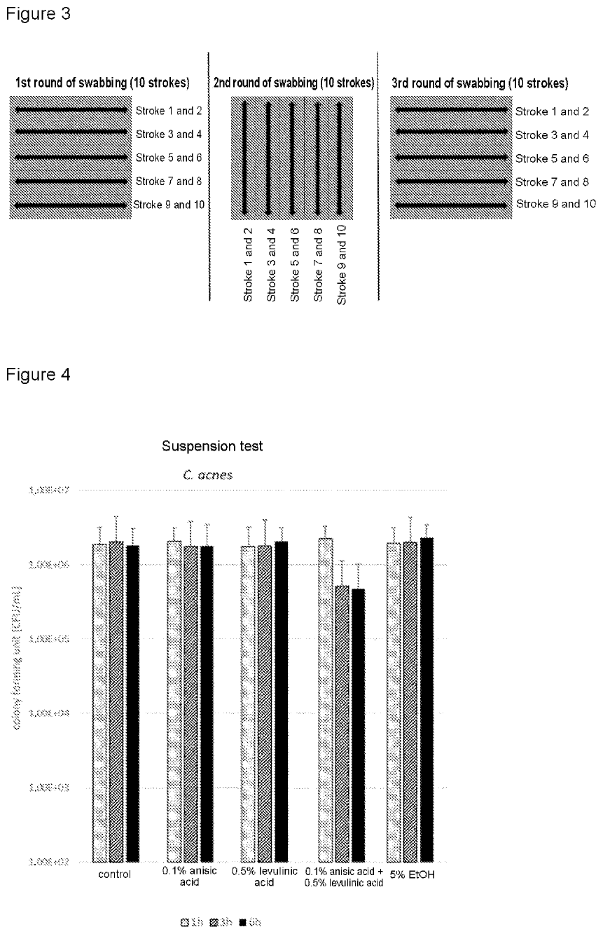

All test subjects went through 3-day preconditioning with a specified wash gel. After rinse-off from the cheeks, “Test formulation 5” as described in Table 1 is applied according to the instructions. Swabbing is then carried out using a Floq swab. 3×10 strokes are swabbed on a 5 cm×5 cm area of the cheek in a defined pattern. The swabbing pattern is depicted in FIG. 3. During the sampling cycles, the Floq swab is soaked in a rinse-off buffer, and after the third round of sampling, the swab remains in the rinse-off buffer. The solution is directly diluted and each sample is plated out on 2 agar plates using a spiral plater. Swabbing is then repeated at the appropriately specified time points, usually after 1 week. Lastly, half of the agar plates were incubated in the absence of oxygen at 37° C. for 5 days and the other half were incubated at 37° C. for 24 h. The agar plates were then evaluated using a Countermat.

The results of the ATA test are shown in FIGS. 1 and 2. FIG. 1 shows the results of the anaerobic culturing. It can be seen that the cell count of the anaerobic bacteria distinctly declined as a result of treatment with the test formulation containing a combination of anisic acid and levulinic acid. Such a reduction in the cell count cannot be seen in FIG. 2, which shows the results of the aerobic culturing. On the contrary, it was possible here to even record an increase in the cell count. FIGS. 1 and 2 therefore impressively show the selective efficacy of the combination of anisic acid and levulinic acid according to the invention.

### Example 3: Suspension Tests

The effect of the formulations according to the invention was further tested in suspension tests with S. epidermidis and C. acnes. Glycerol stock cultures of S. epidermidis (DSM 1798) and C. acnes (DSM 1897), which had been created according to European standard 12353, were stored in a refrigerator at 5° C. These liquid stock cultures were used to prepare a first passage. Here, a sterile disposable inoculation loop was used to streak the relevant culture on an agar plate (C. acnes: COST agar, S. epidermidis: COAST agar) and the culture was then incubated. The cultures of C. acnes were incubated at 37° C. for 5 days, whereas the cultures of S. epidermidis were incubated at 37° C. for 24 h. Said first passage was then used to prepare a second passage by picking of a few colonies using a sterile disposable inoculation loop and subsequent streaking thereof onto a new agar plate. Said second passage was then incubated at the appropriate temperature for 5 days or 24 h. The second passage obtained was then used for the suspension test.

To prepare the suspensions, a few colonies of the second passage were removed using a sterile inoculation loop and added to 10 mL of medium in a baffled flask containing 5 g of glass beads (4 mm diameter) and gently shaken for 3 min. Thereafter, optical density (OD) was used to set the approximate number of cells per mL. Optical density was determined at 600 nm using an Eppendorf photometer. The OD obtained was adjusted to a value around 0.8 (±0.01) and then diluted again 1:50. This dilution strategy yielded a cell count of about 1×10⁶.

For the test formulations, 50 μL of the solution of antimicrobial raw material and 450 μL of medium were initially charged in each case in sterilized Eppendorf reaction tubes (C. acnes: anaerobic medium, S. epidermidis: BHI medium). For the positive control, 500 μL of medium were initially charged. After addition of 500 μL of the prepared culture suspension, 1:10 dilutions of all samples were prepared at different time points (after 1 h, 3 h and 6 h). For the positive control, a dilution was additionally prepared directly after addition of culture (0 min) and plated out using a spiral plater. Throughout the test period, the samples were stored in an Eppendorf shaker at 1000 rpm and 30° C. or 37° C. For the 1:10 dilutions, 900 μL of neutralization medium were initially charged in Eppendorf reaction tubes and 100 μL of the suspension of culture raw material were added thereto at the time points of removal. After the test was completed, all agar plates were placed in an incubator for incubation and evaluated on a Countermat after incubation.

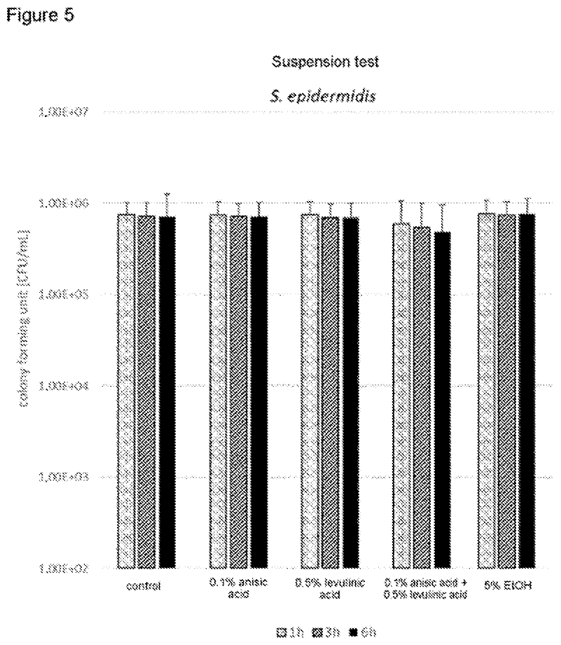

The results are shown in FIGS. 4 and 5. It can be seen that the use of anisic acid, levulinic acid or a combination of both substances does not significantly affect the growth of the aerobic bacterium S. epidermidis. In the case of the anaerobic bacterium C. acnes on the other hand, the sole use of anisic acid or levulinic acid does not lead to a reduction in the cell count. However, the combined use leads to a significant selective antimicrobial effect.

1.-14. (canceled)

15. A composition for specific alteration of the skin flora, wherein the composition comprises:
(i) more than 0.3% (w/w) anisic acid and/or an antimicrobially active salt thereof, and
(ii) more than 1.0% (w/w) levulinic acid and/or an antimicrobially active salt thereof.

16. The composition of claim 15, wherein the composition comprises
(i) at least 0.75% (w/w) anisic acid and/or an antimicrobially active salt thereof, and
(ii) at least 1.5% (w/w) levulinic acid and/or an antimicrobially active salt thereof.

17. The composition of claim 15, wherein the composition comprises
(i) from 0.75% to 5.0% (w/w) anisic acid and/or an antimicrobially active salt thereof, and
(ii) 1.5% to 5.0% (w/w) levulinic acid and/or an antimicrobially active salt thereof.

18. The composition of claim 15, wherein the composition is present as a cream, lotion, gel, ointment, paste or solid stick.

19. The composition of claim 15, wherein the composition contains sufficient amounts of (i) and (ii) to be effective in treating dermatitis.

20. The composition of claim 15, wherein the composition contains sufficient amounts of (i) and (ii) to be effective in treating acne.

21. A method of treating a skin disorder, wherein the method comprises applying to skin affected by the skin disorder the composition of claim 15 in an amount which is effective for treating the skin disorder.

22. The method of claim 21, wherein the skin disorder is acne.

23. A method of selectively reducing the number of anaerobic bacteria on skin, wherein the method comprises applying to the skin a combination of (i) anisic acid and/or an antimicrobially active salt thereof and (ii) levulinic acid and/or an antimicrobially active salt thereof.

24. The method of claim 23, wherein the anaerobic bacteria are bacteria of the genus Cutibacterium.

25. The method of claim 24, wherein the anaerobic bacteria are bacteria of the species Cutibacterium acnes.

26. The method of claim 23, wherein aerobic bacteria on the skin are substantially not reduced.

27. The method of claim 23, wherein the anaerobic bacteria are bacteria of the genus Staphylococcus.

28. The method of claim 27, wherein the anaerobic bacteria are bacteria of the species S. epidermidis.

29. The method of claim 23, wherein the skin is facial skin.

30. The method of claim 23, wherein components (i) and (ii) are applied to the skin simultaneously.

31. The method of claim 23, wherein components (i) and (ii) are applied to the skin consecutively.

32. The method of claim 23, wherein the combination is administered in the form of a composition comprising from 0.01% to 5.0% (w/w) anisic acid and/or an antimicrobially active salt thereof.

33. The method of claim 23, wherein the combination is administered in the form of a composition comprising from 0.01% to 5.0% (w/w) levulinic acid and/or an antimicrobially active salt thereof.

34. The method of claim 32, wherein the combination is administered in the form of a composition comprising from 0.01% to 5.0% (w/w) levulinic acid and/or an antimicrobially active salt thereof.
